(19) United States
(12) Patent Application Publication
(10) Pub. No.: US 2026/0326115 A1
(43) Pub. Date: Oct. 22, 2026

(54) METHODS FOR SEPARATING MULTIPLEXED BEADS

(71) Applicant: DH Technologies Development Pte. Ltd., Singapore (SG)

(72) Inventors: Chang LIU, Richmond Hill (CA); Thomas R. COVEY, Newmarket (CA)

(73) Assignee: DH Technologies Development Pte. Ltd., Singapore (SG)

(21) Appl. No.: 19/115,634

(22) PCT Filed: Sep. 27, 2023
(86) PCT No.: PCT/IB2023/059643

§ 371 Date: Mar. 26, 2025

Related U.S. Application Data
(60) Provisional application No. 63/411,398, filed on Sep. 29, 2022.

(51) Int. Cl. B03C 1/01; B01D 15/38; B03C 1/28

(52) U.S. Cl. CPC B03C 1/01; B01D 15/3804; B03C 1/288; B03C 2201/18; B03C 2201/26

(57) ABSTRACT

A method of processing a liquid sample includes providing the sample. A first set of beads is introduced to the sample. The first set of beads includes a bead characteristic and a first bead performance value. A second set of beads is introduced to the sample. The second set of beads includes the bead characteristic and a second bead performance value different than the first bead performance value. The sample is mixed with the first set of beads and the second set of beads. The first set of beads is captured in a first location within the sample.

### CROSS-REFERENCE TO RELATED APPLICATION

This application is being filed on Sep. 27, 2023, as a PCT International Patent Application that claims priority to and the benefit of U.S. Provisional Application No. 63/411,398, filed on Sep. 29, 2022, which is hereby incorporated by reference in its entirety.

### BACKGROUND

Surface functionalized particles such as beads are used widely in sample processing. They provide convenient automation options for removing, adding, and manipulating reagents in a well-controlled manner. In most assays, a single type of particle with a type of affinity group is used for the target species immobilization. Such particles are also sometimes utilized to mix the sample, introduce analytes of other compounds to the sample, remove target analytes therefrom, etc. Particles may be mixed with various liquids (e.g., samples, wash liquids, other liquids, etc.), at different stages of a process. Each liquid may include one or more binding agents, molecules, analytes, etc.

As used herein, the term “binding agent” refers to a molecule capable of specifically binding a target analyte. A binding agent can be any of a number of different types of molecules, including an antibody or antigen-binding fragment thereof, or other protein, peptide, polysaccharide, lipid, a nucleic acid or nucleic-acid analog, such as an oligonucleotide, aptamer, or PNA (peptide nucleic acids). The term “target analyte” refers to a molecule, compound, or other component in a sample. Target analytes may include but are not limited to peptides, proteins, polynucleotides, organic molecules, sugars and other carbohydrates, and lipids. The term “specific binding” refers to binding of an antibody or other binding agent to an epitope on a cell or target analyte to which the antibody or binding agent is targeted.

### SUMMARY

In one aspect, the technology relates to a method of processing a liquid sample, the method includes: providing the sample; introducing a first set of beads to the sample, wherein the first set of beads includes a bead characteristic and a first bead performance value; introducing a second set of beads to the sample, wherein the second set of beads includes the bead characteristic and a second bead performance value different than the first bead performance value; mixing the sample with the first set of beads and the second set of beads; and capturing the first set of beads in a first location within the sample. In an example, the method further includes capturing the second set of beads in a second location within the sample. In another example, the first bead characteristic includes at least one of the following: a bead magnetic response; a bead density; a bead surface affinity; a bead surface hydrophobicity; and a bead size. In yet another example, the second bead characteristic includes a same bead characteristic as the first bead characteristic. In still another example, wherein mixing the sample includes flowing the sample, the first set of beads, and the second set of beads through a sample conduit; and wherein capturing the first set of beads includes applying a first magnetic field to the flowing sample, the first set of beads, and the second set of beads, wherein the applied first magnetic field is adjusted to attract the first set of beads.

In another example of the above aspect, the method further includes adjusting a flow rate of the flowing sample based at least in part on at least one of a bead magnetic response, and a strength of the applied first magnetic field. In an example, applying the first magnetic field includes applying the first magnetic field at a first location along the sample conduit. In another example, the method further includes terminating application of the first magnetic field. In yet another example, the method further includes collecting the first set of beads separate from the second set of beads. In still another example, the method further includes disposing the sample, the first set of beads, and the second set of beads in a sample container; and wherein mixing the sample includes agitating the sample; and wherein capturing the first set of beads from the sample includes applying a first magnetic field to the sample, wherein the applied first magnetic field is adjusted to attract the first set of beads.

In another example of the above aspect, agitating the sample includes at least one of activating a mixer in the sample, shaking the sample, and aspirating the sample with a pipette. In an example, applying the first magnetic field includes inserting a first magnet into the sample. In another example, applying the first magnetic field includes disposing a first magnet adjacent the sample. In yet another example, the first magnetic field is disposed at a first elevation above a bottom surface of the sample container, and wherein the first set of beads includes at least one of: a bead magnetic response greater than a bead magnetic response of the second set of beads, and a bead density less than a bead density of the second set of beads. In still another example, the method further includes disposing the sample, the first set of beads, and the second set of beads in a sample container; and wherein capturing the first set of beads includes disposing a surface including immobilized affinity tags in the sample container, wherein the surface is configured to generate an affinity interaction with the first set of beads.

In another example of the above aspect, the method further includes disposing the sample, the first set of beads, and the second set of beads in a sample container; and wherein capturing the first set of beads includes disposing a surface in the sample container, wherein the surface is configured to generate an interaction with the first set of beads based at least in part on at least one of a bead surface covalent interaction and a bead surface coating charge interaction. In an example, the surface is disposed within the sample container. In another example, capturing the first set of beads includes inserting the surface into the sample container. In yet another example, capturing the first set of beads includes introducing an aqueous solution to the sample and where a hydrophilicity of the first set of beads draws the first set of beads to the aqueous solution. In still another example, capturing the first set of beads includes introducing a filter to the sample, wherein the filter is configured to capture the first set of beads, wherein the first set of beads includes a bead size greater than a bead size of the second set of beads.

In another example of the above aspect, capturing the first set of beads includes passing the sample through a filter configured to capture the first set of beads, wherein the first set of beads includes a bead size greater than a bead size of the second set of beads.

### BRIEF DESCRIPTION OF THE DRAWINGS

FIG. 1A is a schematic depiction of bead characteristic types that may be combined into a single bead.

FIG. 1B depicts a method of processing a liquid sample.

FIG. 2A-2C depicts a method of redistributing beads having different magnetic response performance values in a liquid sample.

FIGS. 3A-3C depicts another method of redistributing beads having different magnetic response performance values in a liquid sample.

FIGS. 4A-4C depicts a method of redistributing beads having different density performance values in a liquid sample.

FIGS. 5A-5C depicts a method of redistributing beads having different charge affinity performance values in a liquid sample.

FIGS. 6A-6C depicts a method of redistributing beads having different covalent affinity performance values in a liquid sample.

FIGS. 7A-7C depicts a method of redistributing beads having different hydrophobicity performance values in a liquid sample.

FIGS. 8A-8C depicts a method of redistributing beads having different size performance values in a liquid sample.

FIGS. 9A-9C depicts a method of redistributing beads having different size performance values in a liquid sample.

FIG. 10A-10C depicts a method of redistributing beads having multiple different characteristic performance values in a liquid sample.

### DETAILED DESCRIPTION

Very small beads or particles are used in biochemical analysis to assist with automating assay protocols in a laboratory, while maintaining sensitivity and accuracy. In examples, such beads are manufactured on a microscale or nanoscale. In examples, beads may be manufactured from or packed with magnetic material. In other examples, beads may be coated with binding agents to bind with target analytes a sample. In still other examples, the coating may release reagents or molecules into the sample, or bind to target analytes therein, otherwise enable reactions therein.

For more complex processes, multiple types of coatings, as well as multiple types of magnetic particles, could be used in the same assay. For example, multiple surface functional groups may be co-immobilized on the same beads. Examples thereof include beads having both Streptavidin and Trypsin coatings, such as manufactured by Perfinity Biosciences of West Lafayette, Indiana. Multiple types of beads may be introduced into a sample, each bead type having a particular surface group. For example, in the SEER workflow, multiple types of particles may be added in the same sample for target extraction. In other examples, multiple types of magnetic particles, each having different magnetic responses, were utilized. The high-response beads were added into the sample as the “stir bars” and were mixed with a surface-coated low-response particles, on an electromagnetic mixer.

The technologies described herein utilize multiple sets of beads having the same predetermined characteristic, but different performance values thereof, in a single sample. More specifically, each bead in a single set of beads would display a particular characteristic and a same performance valve of that characteristic. Different sets of beads would differ from each other based on the performance valves of the beads in the different sets. In this context, the term “characteristic” means a predefined quality of the bead, such as a material property, a coating property, or other property by which the bead may be evaluated. Examples of such characteristics are described in more detail below and include, but are not limited to, a bead magnetic response, a bead density, a bead surface affinity, a bead surface hydrophobicity, and a bead size. As used herein, the term “performance value” means a value that differentiates one set of beads defined by a particular characteristic from another set of beads having the same particular characteristic. “Performance value” is not necessarily absolute; rather it is used herein as a relative term to compare the performance of multiple sets of beads having the same characteristic. For example, a first set of beads having a high density has a different performance value than a set of beads having a low density, even though both sets of beads are defined by the same characteristic: density. Similarly, a first set of beads coated with analytes that will make them bond to a predetermined receptor on a substrate has a different performance value than a second set of beads that lacks such specific bonding analytes, even though both sets of beads are defined by the same characteristic: surface affinity.

By using sets of beads that perform differently (based on their respective performance values), the sets of beads may be separated from each other discretely. In an example, this separation may be a change in distribution within a sample (e.g., a set of beads may be isolated in a discrete area of a sample container). In another example, one set of beads may be removed from a sample while the other set of beads remains therein. In another example, all beads may be removed from a sample and then separated thereafter. This ability to redistribute or separate beads may enable more complex processes to be performed. For example, if two sets of beads are coated with different analytes, one of which requires a longer reaction time to ensure proper target bonding, the beads with the shorter processing time may be removed discretely from the beads with the longer processing time, which may remain in the sample. Bead characteristics may vary as required or desired for a particular application. At a minimum, two sets of beads displaying the same characteristic (but different performance values thereof) may be used in a single process or sample. However, more than two sets of beads (each with a different performance value of the same characteristic) may be utilized in a single sample or process. Further, single set of beads may also be defined by multiple (e.g., two, three, four, five, or more characteristics), each of which may be leveraged in a particular process. Examples of such beads are described herein, but may include beads that are defined by both a surface affinity characteristic and a density characteristic. For clarity herein, processes involving the use of two sets of beads displaying different performance values of the same characteristic in a single sample are described.

For further clarification, example characteristics and example performance values of each characteristic, are described below:
Bead magnetic response. Beads may be manufactured from materials, or packed with materials, that display a magnetic response characteristic. In examples, performance values of this magnetic response characteristic may be described as a high magnetic response and a low magnetic response. Beads having a high magnetic response may be redistributed within a sample based on the application of a magnetic or electromagnetic force that is optimized, adjusted, or otherwise controlled to target the high magnetic response beads. As used herein, the terms “high magnetic response” and “low magnetic response” are relative between two sets of beads. Depending on the number of sets of beads utilized with a single sample, beads described by other relative terms such as “medium magnetic response” may also be utilized.
Bead density. Beads may be made of materials that display a density characteristic. Sets of beads may be made of material having different densities or may be manufactured of the same material, but to different densities. Beads having higher densities may self-locate within different portions of a sample container. For example, low-density beads may locate proximate an upper volume or surface of a container, while higher density leads may settle towards a lower volume thereof. This change in distribution would allow the lower density beads to be removed via pipetting from the sample, while the higher density beads remain.
Bead surface affinity. Beads may be coated with materials that display a surface affinity characteristic, which enable interactions with constituents within a sample, for example. Affinity interactions may be in the form of a covalent interaction or a charge interaction. In the former interaction, the bead coating may include reagents or other receptors that are drawn towards a corresponding attachment point on a surface that may be disposed in or introduced to a sample. In the latter interaction, positively or negatively charged targets on a bead coating may be drawn to an oppositely-charged contact on a surface. If disposed in a sample container or vessel, forces generated by the affinity integrations may draw the appropriate beads thereto for removal, or to simply isolate those beads away from a particular volume portion of the sample.
Bead surface hydrophobicity. Beads may be coated with materials that display a surface hydrophobicity characteristic. A first set of beads may be coated with a high hydrophilicity coating while another set of beads may be coated with a high hydrophobicity coating. The different sets of beads may be introduced to an organic sample and mixed. Thereafter, an aqueous solution may be introduced to the sample, and the high hydrophilicity beads are drawn thereto for redistribution and/or removal.
Bead size exclusion. Beads may be manufactured to certain size characteristics. Once adequately mixed in a sample, a filter of a desired or required porousness may be introduced to the sample, or the sample flowed therethrough, where the target (typically larger) beads are retained in the filter for separation and removal.

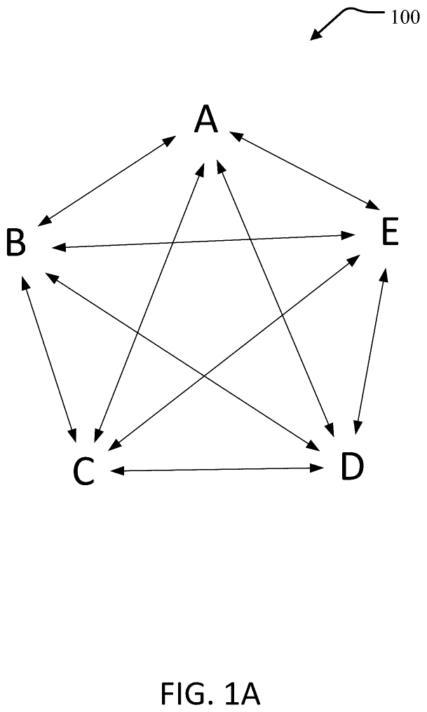

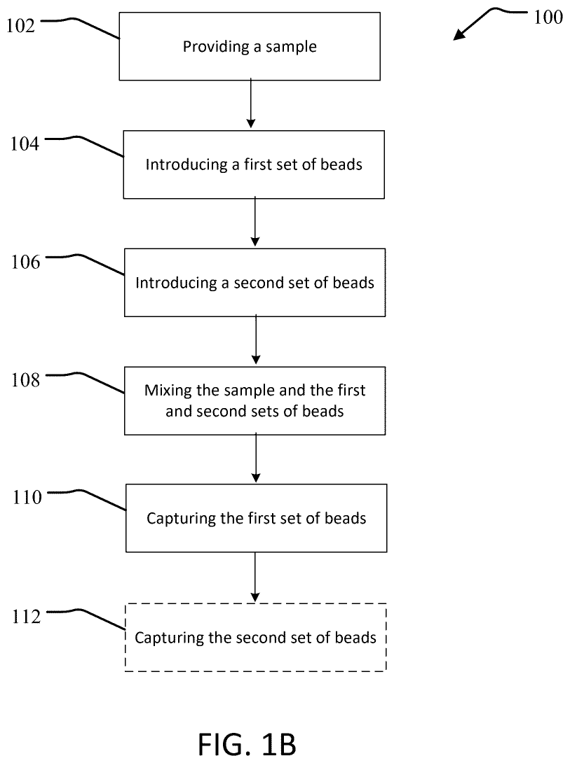

Beads defined by multiple characteristics are also contemplated. Such beads may be used in processes where multiple separations from other sets of beads are required. Beads displaying a desired number of one, two, three, four, or five of the above-identified characteristics are specifically contemplated herein. FIG. 1A schematically illustrates this concept, where a single bead may be defined by one or more particular characteristic(s). In FIG. 1A, bead magnetic response is depicted by the letter “A”, bead density is depicted by the letter “B”, bead surface affinity is depicted by the letter “C”, bead surface hydrophobicity is depicted by the letter “D”, and bead size is depicted by the letter “E”. The double-headed arrows indicate that a bead having a particular defined characteristic may also be defined by another characteristic type. Further, any number of combinations of characteristics are contemplated. Thus, a bead may be defined by one or more characteristic(s). In the Examples below, sets of beads of the same characteristic are labeled with the same prefix consistent with the above (e.g., A, B, C, D, E). Different suffix labels indicate different performance values of that characteristic (e.g., a bead set labeled “D1” would have a different hydrophobicity than a bead set labeled “D2”).

FIG. 1B depicts a method 100 of processing a liquid sample. Various Examples consistent with the method 100 are described in more detail below. The method 100 begins with operation 102, providing the sample. The sample may be in a flowing condition through a conduit or may be in a static condition in a container, vessel, test tube, well plate, etc. In operation 104, a first set of beads is introduced to the sample. This first set of beads may be defined by a first bead characteristic and a first performance value, such as described above. In operation 106, a second set of beads is introduced to the sample. This second set of beads displays a second bead performance value that is different than the first bead performance value, but of the same characteristic. In operation 108, the first set of beads and the second set of beads are mixed in the sample. Different modes of mixing are described below with regard to the various Examples. Thereafter, the first set of beads may be captured, as depicted in operation 110. In optional operation 112, the second set of beads may also be captured (e.g., in a second location within the sample). In the Examples below, different operations of the method 100 are referenced in different parts of the descriptions of each Example.

### EXAMPLE 1

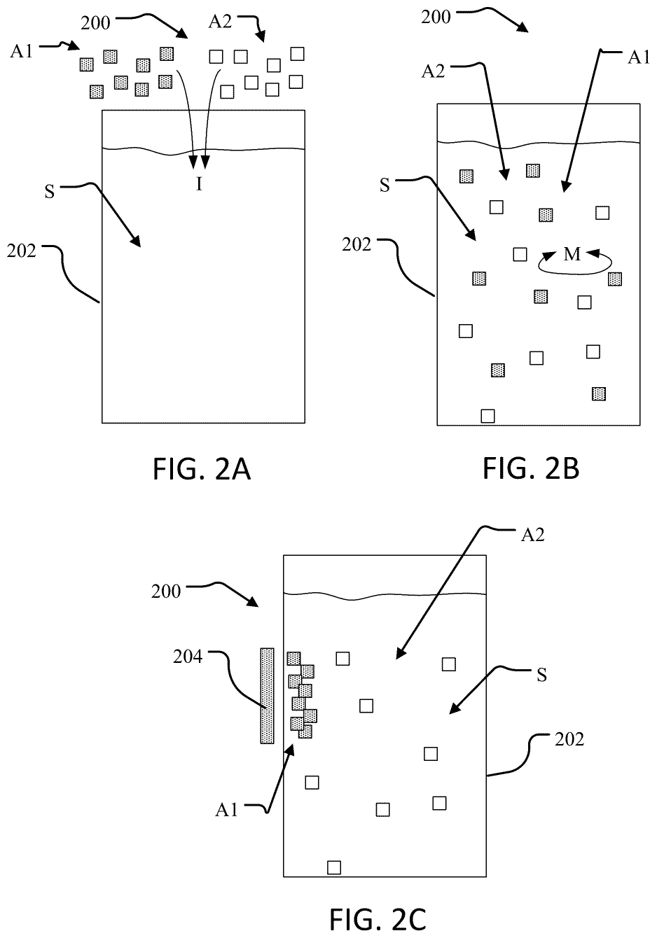

FIGS. 2A-2C depicts a method 200 of distributing beads A1, A2 having different magnetic response performance values in a liquid sample. A container 202 contains a sample S, as depicted in FIG. 2A (operation 102). A first set of beads A1 having, in this case, a high magnetic response performance value is introduced I to the sample S (operation 104). Additionally, a second set of beads A2 having a low magnetic response performance value is introduced I to the sample S (operation 106). The introductions I may be performed contemporaneously, simultaneously, or at entirely different times. In FIG. 2B, the sample S, along with the sets of beads A1, A2, are mixed M (operation 108). This mixing M may be performed by agitating the container 202, or by mixing the sample S directly with a mechanical mixer or a magnetic mixer. In an example, the high magnetic response beads A1 may be utilized for the magnetic mixing process, as known in the art. In another example, the sample S and beads A1, A2 may be mixed by aspirating the sample S, e.g., with a pipette. The mixing M process generally evenly distributes the sets of beads A1, A2 within the sample S, and further enhances any required interactions between the sample S and any coatings having receptors that may be present on the beads A1, A2. In FIG. 2C, a magnet 204 is disposed adjacent the container 202; in examples, the magnet 204 may be the same magnet used to perform the magnetic stirring function. The magnet 204 may be placed relative to the container 202 as required or desired for a particular application. The magnet 204 may be a permanent magnet or an electromagnet and may be optimized to attract the high magnetic response beads A1. This attraction redistributes the sets of beads A1, A2 within the sample S. More specifically, the high magnetic response beads A1 are draw towards the location of the magnet 204 (operation 110), while the low magnetic response beads A2 remain distributed in the sample. In other examples, a magnet may be inserted into the sample S, which would draw the high magnetic response beads A1 to the magnet for removal. Regardless, once the beads A1 are redistributed, the sample S (and potentially some of the beads A2) may be drawn from the sample for further processing.

### EXAMPLE 2

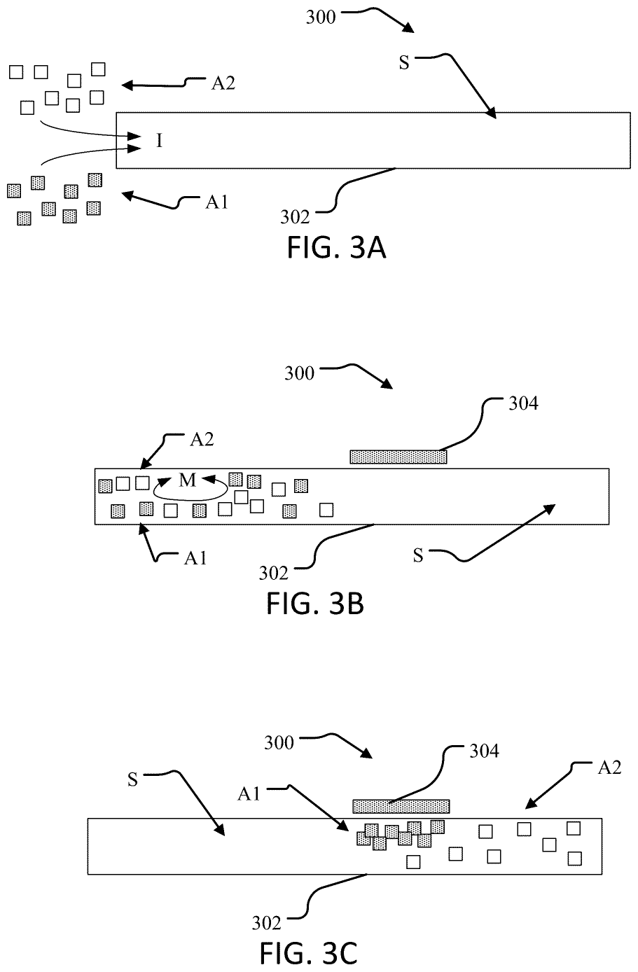

FIGS. 3A-3C depicts a method 300 of distributing beads A1, A2 having different magnetic response performance values in a liquid sample. A sample S flows through a container 302, in this a conduit, as depicted in FIG. 3A (operation 102). A first set of beads A1 having, in this case, a high magnetic response is introduced I to the sample S (operation 104) so as to enter into the conduit 302. Additionally, a second set of beads A2 having a low magnetic response performance value is introduced I to the sample S (operation 106). The introductions I may be performed contemporaneously, simultaneously, or at entirely different times. In FIG. 3B, the sample S, along with the sets of beads A1, A2, are mixed M (operation 108). This mixing M may be performed by passing the sample S and beads A1, A2 through a portion of the conduit 302 characterized by physical agitation. Other methods of mixing M may be utilized, for example by mixing the sample S directly with a mechanical mixer or a magnetic mixer, or by introducing the sample S in such a way so as to achieve turbulent flow conditions. In an example, the high magnetic response beads Al may be utilized for a magnetic mixing process, as known in the art. The mixing M process generally evenly distributes the sets of beads A1, A2 within the sample S, and further enhances any required interactions between the sample S and any coatings having receptors that may be present on the beads A1, A2. In FIG. 3C, a magnet 304 is disposed adjacent the container 302 as the sample S flows past. In examples, the magnet 304 may be the same magnet used to perform a magnetic stirring function. The magnet 304 may be placed relative to the container 302 as required or desired for a particular application. In examples, the magnet 304 may be disposed at a location sufficiently distant from a mixing location of sample S so as to ensure adequately mixing of the beads A1, A2 prior to contact with the magnetic field. The magnet 304 may be a permanent magnet or an electromagnet and may be optimized to attract the high magnetic response beads A1. This attraction redistributes the sets of beads A1, A2 within the sample S. More specifically, the high magnetic response beads A1 are drawn towards the location of the magnet 304 (operation 110), while the low magnetic response beads A2 may continue to flow within the conduit 302. Other process parameters may be selected as required or desired to enhance capture of the beads A1 at the magnet 304. Such parameters include, but are not limited to: sample viscosity, sample flow rate, magnetic field strength, and other parameters as would be apparent to a person of skill in the art. In other examples, a magnet may be inserted into the conduit 302, such that the sample S passes around it, which would draw the high magnetic response beads A1 to the magnet for removal. Regardless, once the beads A1 are redistributed, the sample S (and potentially some of the beads A2) may continue to flow through the conduit 302 and on to further processing. In the depicted example, the sample S and the low magnetic response beads A2 flow past the magnet 304 so as to separate the beads A1 from the beads A2 within the sample S. In another example, the magnetic field may be maintained once the beads A2 have flowed past (e.g., for collection thereof downstream). Thereafter, the magnetic field may be terminated so as to release the beads Al from the captured condition of FIG. 3C, so they may flow downstream to be separately collected.

### EXAMPLE 3

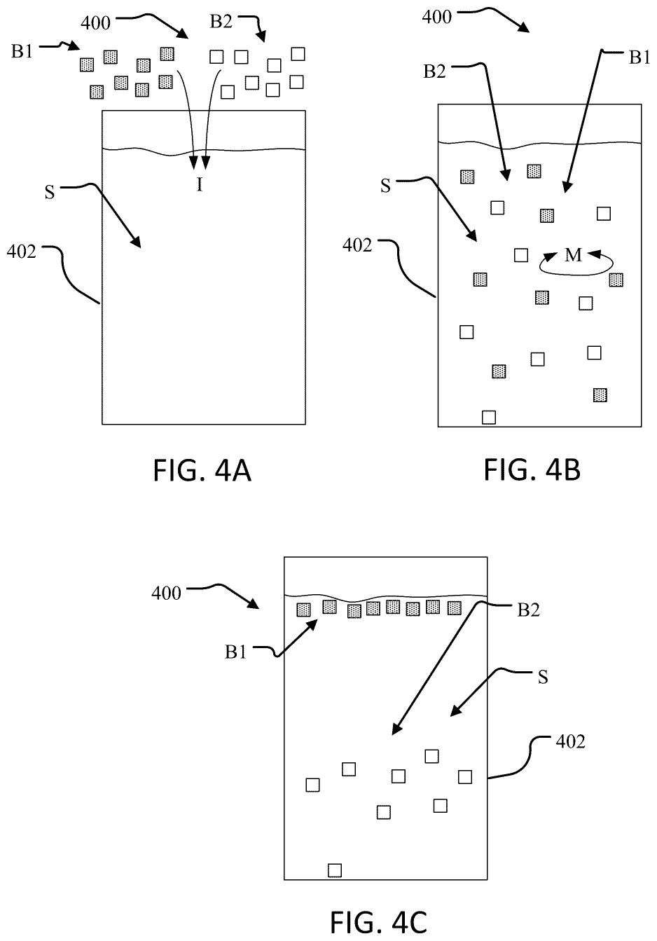

FIGS. 4A-4C depict a method 400 of distributing beads B1, B2 having different density performance values in a liquid state. A container 402 contains a sample S, as depicted in FIG. 4A (operation 102). A first set of beads B1 having, in this case, a high-density performance value, is introduced I to the sample S (operation 104). Additionally, a second set of beads B2 having a low-density performance value, is introduced I to the sample S (operation 106). The introductions I may be performed contemporaneously, simultaneously, or at entirely different times. In FIG. 4B, the sample S, along with the sets of beads B1, B2, are mixed M operation 108). This mixing M may be performed by agitating the container 402, or by mixing the sample S directly with a mechanical mixer or a magnetic mixer. The mixing M process generally evenly distributes the sets of beads B1, B2 within the sample S, and further enhances any required interactions between the sample and any coatings having receptors that may be present on the beads B1, B2. In FIG. 4C, mixing M process is terminated; as such, the beads B1 having the lower density are redistributed within the sample S by floating near an upper surface of the sample S. Meanwhile, the high-density beads B2 may remain distributed within the sample S. In other examples, the high-density beads B2 may be of an even higher density, so as to sink within the container 402. Once the beads B1 are redistributed, they may be removed (e.g., via skimming or other processes) from the sample (operation 110), or a pipette may be inserted into the sample to a location lower than the low-density beads B1, and a portion of the sample S may be drawn into the pipette.

### EXAMPLE 4

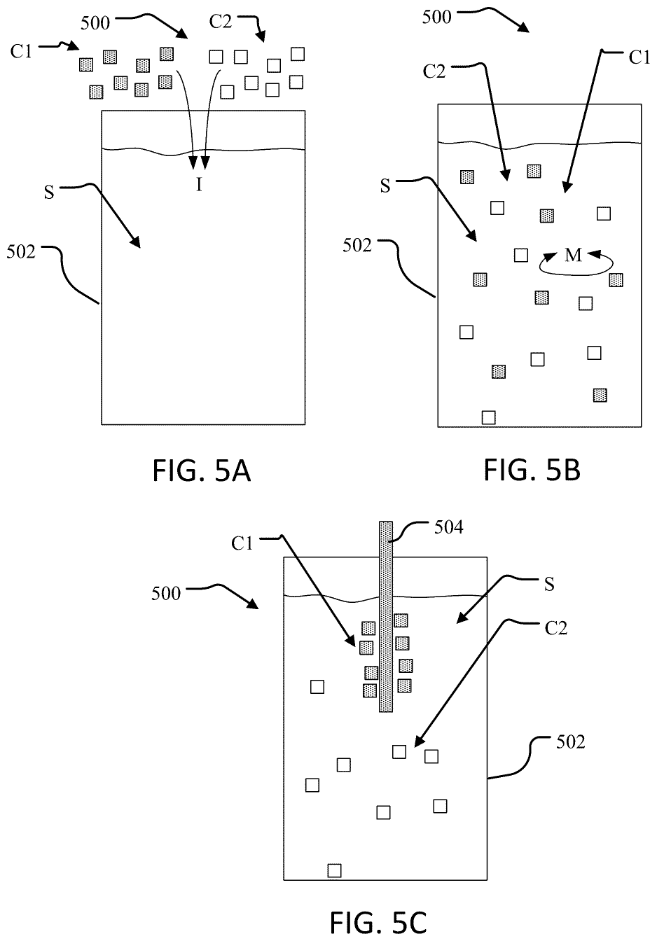

FIGS. 5A-5C depict a method 500 of distributing beads C1, C2 having different surface affinity performance values in a liquid sample. In this Example, the affinity is a charge affinity. A container 502 contains a sample S, as depicted in FIG. 5A (operation 102). A first set of beads C1 having, in this case, a positive charge is introduced I to the sample S (operation 104). Additionally, a second set of beads C2 having a neutral charge is introduced I to the sample S (operation 106). The introductions I may be performed contemporaneously, simultaneously, or at entirely different times. In FIG. 5B, the sample S, along with the sets of beads C1, C2, are mixed M (operation 108). This mixing M may be performed by agitating the container 502, or by mixing the sample S directly with a mechanical mixer or a magnetic mixer. The mixing M process generally evenly distributes the sets of beads C1, C2 within the sample S, and further enhances any required interactions between the sample S and any coatings having receptors that may be present on the beads C1, C2. In FIG. 5C, a substrate 504 is disposed within the container 502 and in this case is negatively charged, e.g., opposite the charge state of the positively charged set of beads C1. As such, the substrate attracts thereto the positively charged beads C1 (operation 110). This attraction redistributes the sets of beads C1, C2 within the sample S. More specifically, the positively charged beads C1 are drawn towards the location of the substrate 504, while the uncharged beads C2 remain distributed within the sample S. Once the beads C1 are redistributed, the sample S (and potentially some of the beads C2) may be drawn from the sample for further processing. In another example, the substrate now bearing the positively charged beads may be removed from the sample S.

### EXAMPLE 5

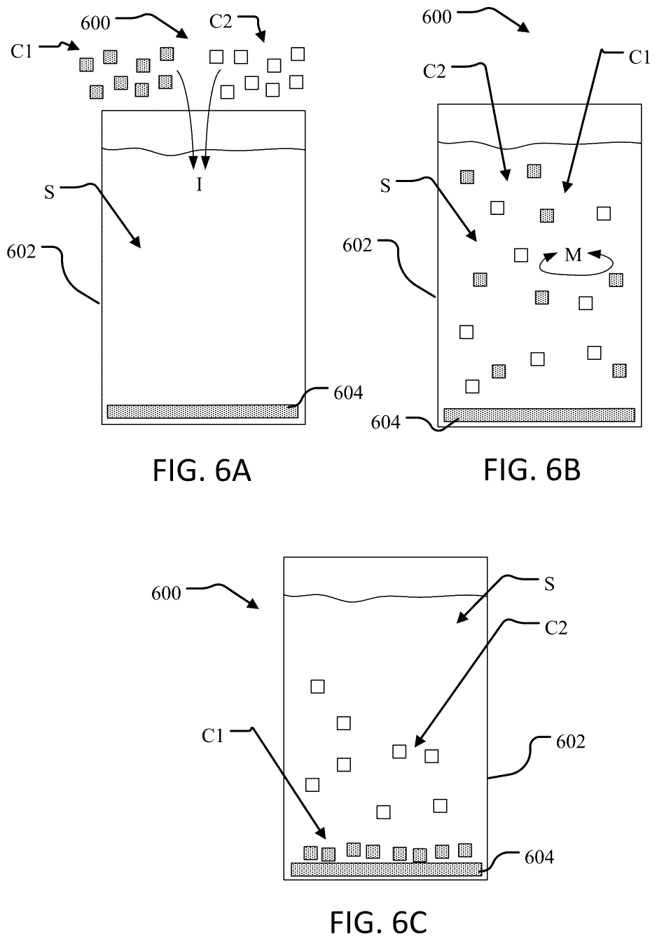

FIGS. 6A-6C depict a method 600 of distributing beads C1, C2 having different surface affinity performance values in a liquid sample. In this Example, the affinity is a covalent affinity. A container 602 contains a sample S, as depicted in FIG. 6A (operation 102). A first set of beads C1 may be coated with a coating containing a ligand having an affinity for a target molecule and is introduced I to the sample S (operation 104). Additionally, a second set of beads C2 which may be defined by an absence of the same ligand is introduced I to the sample S (operation 106). The introductions I may be performed contemporaneously, simultaneously, or at entirely different times. In FIG. 6B, the sample S, along with the sets of beads C1, C2, are mixed M (operation 108). This mixing M may be performed by agitating the container 602, or by mixing the sample S directly with a mechanical mixer or a magnetic mixer. The mixing M process generally evenly distributes the sets of beads C1, C2 within the sample S, and further enhances any required interactions between the sample S and any coatings having receptors that may be present on the beads C1, C2. In FIG. 6C, a substrate 604 is disposed within the container 602, for example, secured to a discrete portion thereof. The substrate 604 in this case is coated with the target molecule for which the ligand in the first set of beads C1 has an affinity. As such, the substrate attracts thereto the liquid affinity beads C1. This attraction redistributes the sets of beads C1, C2 within the sample S such that the beads C1 are draw towards the location of the substrate 604, while the beads C2 remain distributed within the sample S (operation 110). Once the beads C1 are redistributed, the sample S (and potentially some of the beads C2) may be drawn from the sample S for further processing. In another example, the substrate 504 now bearing the beads C1 may be removed from the sample S.

### EXAMPLE 6

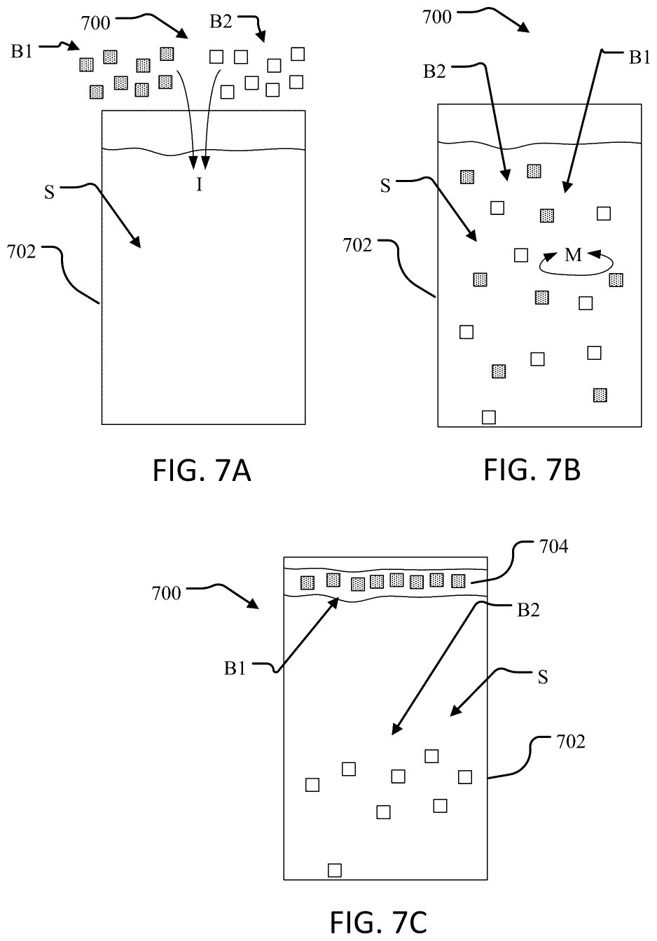

FIGS. 7A-7C depict a method 700 of distributing beads D1, D2 having different hydrophilicity performance values in a liquid state. A container 702 contains a sample S, as depicted in FIG. 7A (operation 102). A first set of beads D1 having, in this case, a high hydrophilicity performance value, is introduced I to the sample S (operation 104). Additionally, a second set of beads D2 having a high hydrophilicity performance value, is introduced I to the sample S (operation 106). The introductions I may be performed contemporaneously, simultaneously, or at entirely different times. In FIG. 7B, the sample S, along with the sets of beads D1, D2, are mixed M (operation 108). This mixing M may be performed by agitating the container 702, or by mixing the sample S directly with a mechanical mixer or a magnetic mixer. The mixing M process generally evenly distributes the sets of beads D1, D2 within the sample S, and further enhances any required interactions between the sample S and any coatings having receptors that may be present on the beads D1, D2. In FIG. 7C, mixing M process is terminated and an aqueous solution 704 is introduced to the sample S, with which it is unable to mix readily. The high hydrophilicity beads D1 are redistributed within the sample by being drawn towards the aqueous solution 704 that settles above the sample (operation 110). Meanwhile, the high hydrophilicity beads D2 may remain distributed within the sample S. Once the beads D1 are redistributed, they may be removed (e.g., via skimming or removed along with a removal of the aqueous solution 704) from the sample S, or a pipette may be inserted into the sample S to a location lower than the high hydrophobicity beads D1, and a portion of the sample S may be drawn into the pipette.

### EXAMPLE 7

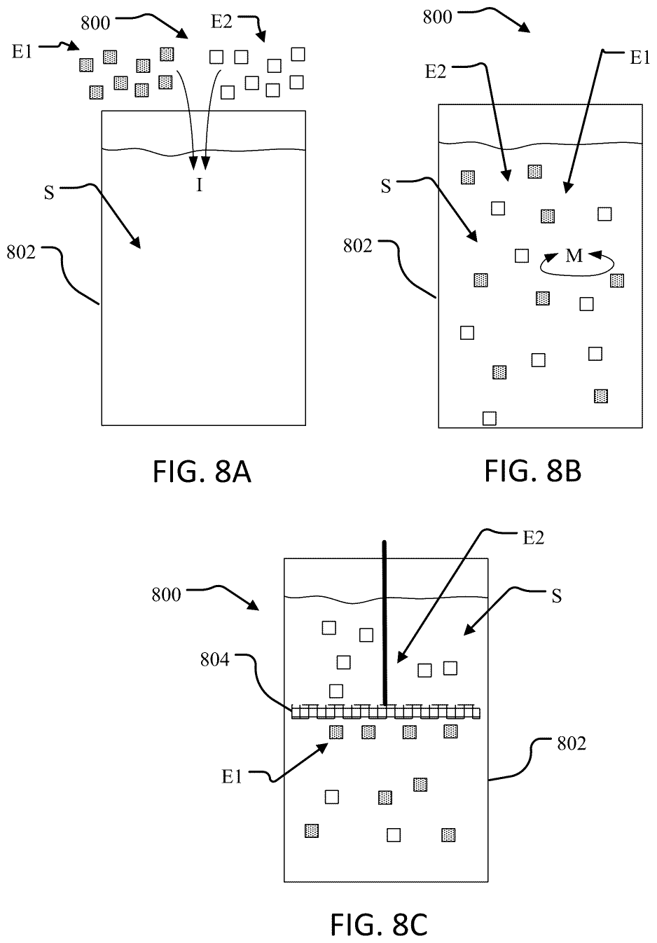

Example 7, FIGS. 8A-8C depict a method 800 of distributing beads E1, E2 having different size performance values in a liquid sample. A container 802 contains a sample S, as depicted in FIG. 8A (operation 102). A first set of beads E1 having, in this case, a larger size performance value is introduced I to the sample S (operation 104). Additionally, a second set of beads E2 having a smaller size performance value is introduced I to the sample S (operation 106). The introductions I may be performed contemporaneously, simultaneously, or at entirely different times. Here, a size performance value may be a measure of diameter, width, or other relevant measurable dimension, as known in the art. In FIG. 8B, the sample S, along with the sets of beads E1, E2, are mixed M (operation 108). This mixing M may be performed by agitating the container 802, or by mixing the sample S directly with a mechanical mixer or a magnetic mixer. The mixing M process generally evenly distributes the sets of beads E1, E2 within the sample S, and further enhances any required interactions between the sample S and any coatings having receptors that may be present on the beads E1, E2. In FIG. 8C, a filter 804 is introduced to the container 802; in examples, the filter 804 may be actuated in a downward direction in the container 802. The filter 804 is defined by a porosity that allows passage of the smaller size beads E2 and retention of the larger size beads E1. This causes a separation of the beads E1, E2 within the sample S, such that the larger size beads E1 are captured or otherwise prevented from passing through the filter 804 (operation 110). As the filter 804 moves further downward, the larger size beads El are trapped below the filter 804, while the smaller size beads E2 are able to pass therethrough. Regardless, once the beads E1 are redistributed, the sample S (and potentially some of the beads E2) may be drawn from the sample S for further processing. In another example, the filter may be present in the container 802 (e.g., disposed near the bottom thereof) and lifted from the container 802 subsequent to the mixing M operation. Beads larger than the porosity of the filter may be captured and removed from the sample S.

### EXAMPLE 8

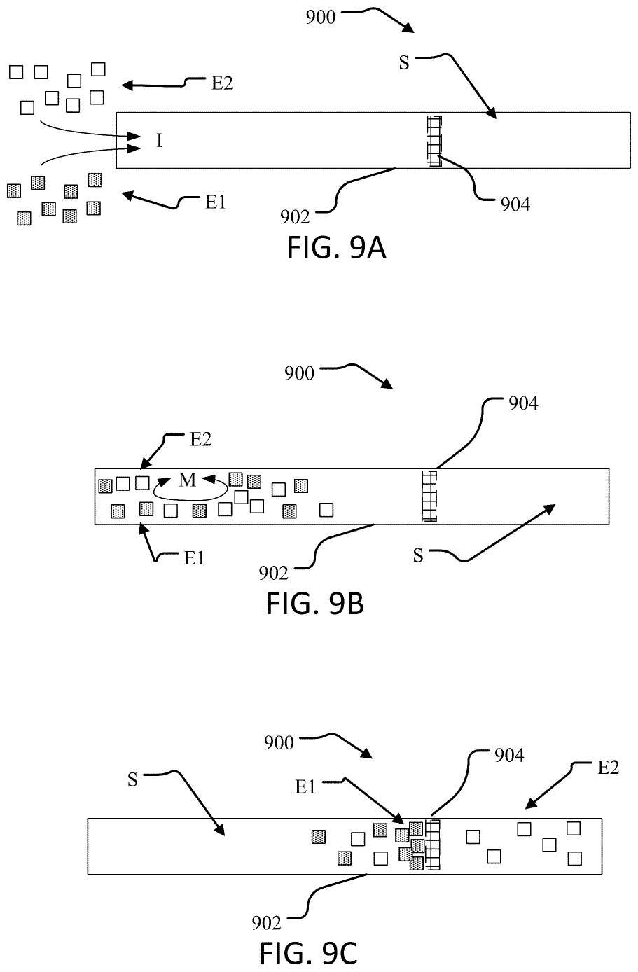

FIGS. 9A-9C depict a method 900 of distributing beads E1, E2 having different size performance values in a liquid state. A sample S flows through a container 902, in this a conduit, as depicted in FIG. 9A (operation 102). A first set of beads E1 having, in this case, a larger size performance value is introduced I to the sample S (operation 104) so as to enter into the conduit 902. Additionally, a second set of beads E2 having a smaller size performance value is introduced I to the sample S (operation 106). The introductions I may be performed contemporaneously, simultaneously, or at entirely different times. In FIG. 9B, the sample S, along with the sets of beads E1, E2, are mixed M (operation 108). This mixing M may be performed by passing the sample S and beads E1, E2 through a portion of the conduit 902 characterized by physical agitation. Other methods of mixing M may be utilized, for example by mixing the sample S directly with a mechanical mixer or a magnetic mixer, or by introducing the sample S in such a way so as to achieve turbulent flow conditions. The mixing M process generally evenly distributes the sets of beads E1, E2 within the sample S, and further enhances any required interactions between the sample S and any coatings having receptors that may be present on the beads E1, E2. A filter 904 is disposed within the container 902 as the sample S flows past. The larger size beads E1 are trapped by the filter 904, while the smaller size beads E2 are able to pass therethrough, as depicted in FIG. 9C (operation 110).

### EXAMPLE 9

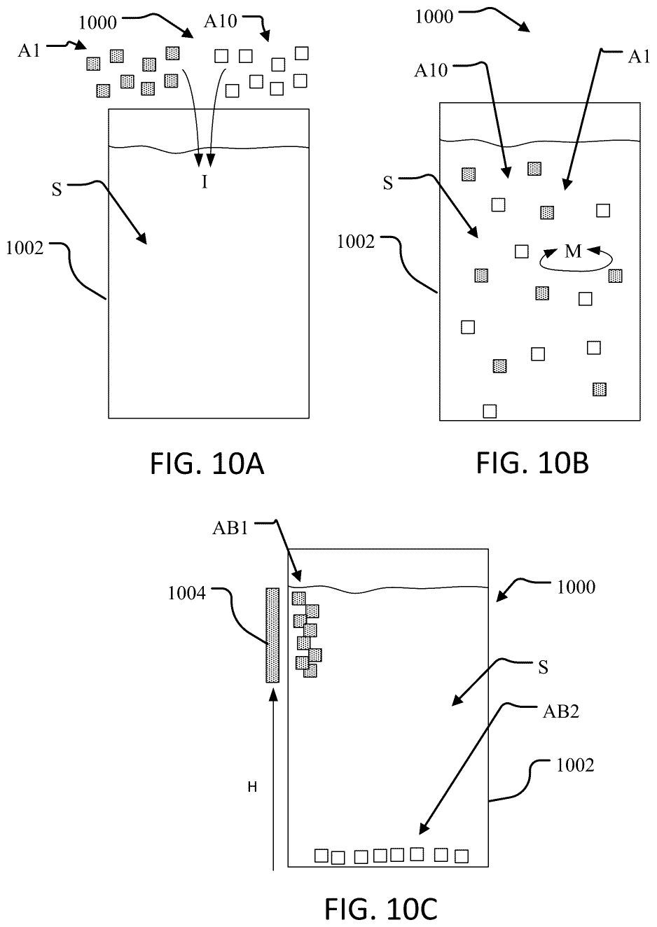

FIGS. 10A-10C depict a method 1000 of distributing beads AB1, AB2 having multiple characteristic performance values in a liquid sample. In this Example 9, a set of beads AB1 is characterized by a magnetic response performance value “A” and a density performance value “B” different than the magnetic response performance value “A” and density performance value “B” of set of beads AB2. More specifically, set of beads AB1 has a magnetic response performance value higher than the magnetic response performance value of set of beads AB2. Further, set of beads AB1 has a density performance value less than that of the set of beads AB2. A container 1002 contains a sample S, as depicted in FIG. 10A (operation 102). The first set of beads, AB1 is introduced I to the sample S (operation 104), as is the second set of beads AB2 (operation 106). The introductions I may be performed contemporaneously, simultaneously, or at entirely different times. In FIG. 10B, the sample S, along with the sets of beads AB1, AB2, are mixed M (operation 108). This mixing M may be performed by agitating the container 1002, or by mixing the sample S directly with a mechanical mixer or a magnetic mixer. In an example, the high-response magnetic beads AB1 may be utilized for the magnetic mixing process, as known in the art. The mixing M process generally evenly distributes the sets of beads AB1, AB2 within the sample S, and further enhances any required interactions between the sample S and any coatings having receptors that may be present on the beads AB1, AB2. In FIG. 10C, a magnet 1004 is disposed adjacent the container 1002, for example at a predetermined height H at or near an upper portion of the container 1002. In examples, the magnet 1004 may be the same magnet used to perform the magnetic stirring function. The magnet 1004 may be a permanent magnet or an electromagnet and may be optimized to attract the high magnetic response beads AB1. This attraction redistributes the sets of beads AB1, AB2 within the sample S. More specifically, the high magnetic response beads AB1 are drawn towards the location of the magnet 1004 (operation 110). Redistribution of the beads AB1, AB2 is further enhanced by the relative differences in densities of the sets of beads AB1, AB2. That is, the set of beads AB2 having the higher density will sink towards the bottom of the container 1002 as depicted. By positioning the magnet 1004 at the height H, significant separation between the sets of beads AB1, AB2 may be achieved. As with other examples, described herein, a magnet may be inserted into the sample S, which would draw the high magnetic response beads to the magnet for removal. Regardless, once the beads AB1, AB2 are redistributed, the sample S may be drawn from the container 1002 for further processing.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

1. A method of processing a liquid sample, the method comprising:
providing the sample;
introducing a first set of beads to the sample, wherein the first set of beads comprises a bead characteristic and a first bead performance value;
introducing a second set of beads to the sample, wherein the second set of beads comprises the bead characteristic and a second bead performance value different than the first bead performance value;
mixing the sample with the first set of beads and the second set of beads; and
capturing the first set of beads in a first location within the sample.

2. The method of claim 1, further comprising capturing the second set of beads in a second location within the sample.

3. The method of claim 1, wherein the first bead characteristic comprises at least one of the following:
a bead magnetic response;
a bead density;
a bead surface affinity;
a bead surface hydrophobicity; and
a bead size.

4. The method of claim 1, wherein the second bead characteristic comprises a same bead characteristic as the first bead characteristic.

5. The method of claim 1:
wherein mixing the sample comprises flowing the sample, the first set of beads, and the second set of beads through a sample conduit; and
wherein capturing the first set of beads comprises applying a first magnetic field to the flowing sample, the first set of beads, and the second set of beads, wherein the applied first magnetic field is adjusted to attract the first set of beads.

6. The method of claim 5, further comprising adjusting a flow rate of the flowing sample based at least in part on at least one of a bead magnetic response, and a strength of the applied first magnetic field.

7. The method of claim 5, wherein applying the first magnetic field comprises applying the first magnetic field at a first location along the sample conduit.

8. The method of claim 5, further comprising terminating application of the first magnetic field.

9. The method of claim 1, further comprising collecting the first set of beads separate from the second set of beads.

10. The method of claim 1, further comprising:
disposing the sample, the first set of beads, and the second set of beads in a sample container; and
wherein mixing the sample comprises agitating the sample; and
wherein capturing the first set of beads from the sample comprises applying a first magnetic field to the sample, wherein the applied first magnetic field is adjusted to attract the first set of beads.

11. The method of claim 10, wherein agitating the sample comprises at least one of activating a mixer in the sample, shaking the sample, and aspirating the sample with a pipette.

12. The method of claim 10, wherein applying the first magnetic field comprises inserting a first magnet into the sample.

13. The method of claim 10, wherein applying the first magnetic field comprises disposing a first magnet adjacent the sample.

14. The method of claim 10, wherein the first magnetic field is disposed at a first elevation above a bottom surface of the sample container, and wherein the first set of beads comprises at least one of:
a bead magnetic response greater than a bead magnetic response of the second set of beads, and
a bead density less than a bead density of the second set of beads.

15. The method of claim 1, further comprising:
disposing the sample, the first set of beads, and the second set of beads in a sample container; and
wherein capturing the first set of beads comprises disposing a surface comprising immobilized affinity tags in the sample container, wherein the surface is configured to generate an affinity interaction with the first set of beads.

16. The method of claim 1, further comprising:
disposing the sample, the first set of beads, and the second set of beads in a sample container; and
wherein capturing the first set of beads comprises disposing a surface in the sample container, wherein the surface is configured to generate an interaction with the first set of beads based at least in part on at least one of a bead surface covalent interaction and a bead surface coating charge interaction.

17. The method of claim 15, wherein the surface is disposed within the sample container.

18. The method of claim 15, wherein capturing the first set of beads comprises inserting the surface into the sample container.

19. The method of claim 1, wherein capturing the first set of beads comprises introducing an aqueous solution to the sample and where a hydrophilicity of the first set of beads draws the first set of beads to the aqueous solution.

20. The method of claim 1, wherein capturing the first set of beads comprises introducing a filter to the sample, wherein the filter is configured to capture the first set of beads, wherein the first set of beads comprises a bead size greater than a bead size of the second set of beads.

21. The method of claim 1, wherein capturing the first set of beads comprises passing the sample through a filter configured to capture the first set of beads, wherein the first set of beads comprises a bead size greater than a bead size of the second set of beads.
